(19) United States
(12) Patent Application Publication
(10) Pub. No.: US 2008/0292572 A1
(43) Pub. Date: Nov. 27, 2008

(54) Production of structured hairstyles using a composition comprising reactive silicone compounds

(76) Inventor: Katarina Benabdillah, le Plessis-Bouchard (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/003,067

(22) Filed: Dec. 19, 2007

Related U.S. Application Data
(60) Provisional application No. 60/878,065, filed on Jan. 3, 2007.

(30) Foreign Application Priority Data
Dec. 20, 2006 (FR) 0655728

(51) Int. Cl. A61K 8/58; A61Q 5/00

(52) U.S. Cl. 424/70.1

(57) ABSTRACT

The present invention relates to the treatment of human keratin fibres to obtain structured hairstyles, for example with structured locks, which consists in applying a composition comprising at least one compound X and Y, at least one of which is a silicone compound, these compounds furthermore being capable of reacting together via a hydrosilylation reaction, or via a condensation reaction, or via a crosslinking reaction in the presence of a peroxide.

The present invention relates to the treatment of human keratin fibres to obtain structured hairstyles, which consists in applying a composition comprising at least one compound X and Y, at least one of which is a silicone compound, these compounds furthermore being capable of reacting together via a hydrosilylation, condensation or crosslinking reaction.

The preparation of certain sophisticated hairstyles consisting of plaits, spikes, curls, buns, etc. is often long and complex and requires in certain cases the intervention of a hairstylist. This is why it is desired to keep such a hairstyle intact for several days, or even longer.

The drawback is that the styling products used to obtain such hairstyles come out on the first shampoo wash and do not allow the hairstyle to be kept for the desired length of time.

In order to overcome this drawback, it may be envisaged to increase the remanence of the product by directly performing a polymerization of certain monomers on the hair. Thus, document U.S. Pat. No. 4,344,763 describes a hair-fixing composition using an aminoalkylalkoxysilane reactive silicone and an ester titanate. It is also known practice to coat the hair using a composition comprising an electrophilic monomer of cyanoacrylate type, especially in patent application FR 2 833 489. Such a composition makes it possible to obtain perfectly coated and non-greasy hair. However, the coating obtained is not entirely satisfactory with regard to external agents such as washing and perspiration. Moreover, the coating obtained is sensitive to fatty substances such as sebum.

The aim of the present invention is to develop a novel hair-treating process that makes it possible to obtain long-lasting fixing while at the same time conserving good cosmetic properties.

One subject of the present invention is thus a process for treating human keratin fibres, such as the hair, to obtain structured hairstyles, in which a composition comprising at least one compound X, at least one compound Y is applied, at least one of the compounds X or Y being a silicone compound, the said compounds X and Y being capable of reacting together via a hydrosilylation reaction, or via a condensation reaction, or via a crosslinking reaction in the presence of a peroxide.

The process according to the invention makes it possible especially to obtain locks comprising an assembly of at least 10 and more advantageously of at least 50 fibres bonded together. Preferably, the minimum length of the fibres is 10 cm.

The process may also be advantageously used for obtaining shampoo-fast hairstyles on frizzy and relaxed African hair.

Moreover, it has been found that the coating obtained is smooth and uniform and shows excellent adhesion to the hair. In addition, this coating is remanent over several shampoo washes, which makes it possible to keep the hairstyle for longer.

However, other characteristics and advantages of the present invention will emerge more clearly on reading the description and the examples that follow.

It should be noted that, in the text hereinbelow, the limits bounding a range of values are included in that range, unless otherwise indicated.

As has been stated previously, the composition applied to the fibres comprises at least one compound X or at least one compound Y, at least one of which is a silicone compound.

### Compounds X and Y

The term “silicone compound” means a compound comprising at least two organosiloxane units. According to one particular embodiment, compounds X and compounds Y are silicone-based. Compounds X and Y may be amino or non-amino compounds. They may comprise polar groups, which may be chosen from the following groups: —COOH, —COO⁻, —COO—, —OH, —NH₂, —NH—, —NR—, —SO₃H, —SO₃⁻, —OCH₂CH₂—, —O—CH₂CH₂CH₂—, —O—CH₂CH(CH₃)—, —NR₃⁺, —SH, —NO₂, I, Cl, Br, —CN, —PO₄³⁻, —CONH—, —CONR—, —CONH₂, —CSNH—, —SO₂—, —SO—, —SO₂NH—, —NHCO—, —NHSO₂—, —NHCOO—, —OCONH—, —NHCSO— and —OCSNH— with R representing an alkyl group.

According to another embodiment, at least one of the compounds X and Y is a polymer whose main chain is predominantly formed from organosiloxane units.

Among the silicone compounds mentioned below, some may have both film-forming properties and adhesive properties depending, for example, on their proportion of silicone or on whether they are used as a mixture with a particular additive. It is consequently possible to modify the film-forming properties or the adhesive properties of such compounds according to the intended use, and this is in particular the case for the reactive elastomeric silicones said to be “room-temperature vulcanizable”.

Compounds X and Y may react together at a temperature ranging between room temperature and 180° C. Advantageously, compounds X and Y may react together at room temperature (20±5° C.) and atmospheric pressure, advantageously in the presence of a catalyst, via a hydrosilylation reaction or a condensation reaction, or a crosslinking reaction in the presence of a peroxide.

### 1—Compounds X and Y Capable of Reacting Via Hydrosilylation

According to one embodiment, compounds X and Y are capable of reacting via hydrosilylation, this reaction being represented schematically in simple terms as follows:

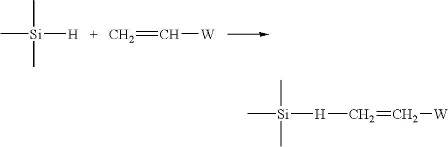

with W representing a carbon-based and/or silicone chain containing one or more unsaturated aliphatic groups.

In this case, compound X may be chosen from silicone compounds comprising at least two unsaturated aliphatic groups. For example, compound X may comprise a silicone main chain whose unsaturated aliphatic groups are pendent on the main chain (side group) or located at the ends of the main chain of the compound (end group). In the rest of the description, these particular compounds will be referred to as polyorganosiloxanes containing unsaturated aliphatic groups.

In this case, compound X may be chosen from silicone compounds comprising at least two unsaturated aliphatic groups.

For example, compound X may comprise a silicone main chain whose unsaturated aliphatic groups are pendent on the main chain (side group) or located at the ends of the main chain of the compound (end group). In the rest of the description, these particular compounds will be referred to as polyorganosiloxanes containing unsaturated aliphatic groups.

According to one embodiment, compound X is chosen from polyorganosiloxanes comprising at least two unsaturated aliphatic groups, for example two or three vinyl or allylic groups, each bonded to a silicon atom.

According to one advantageous embodiment, compound X is chosen from polyorganosiloxanes comprising siloxane units of formula:

$${R}_{m} {R}^{′} {SiO}_{\frac{( 3 - m )}{2}} \qquad ( I )$$

in which:
R represents a linear or cyclic monovalent hydrocarbon-based group containing from 1 to 30 carbon atoms, preferably from 1 to 20 and better still from 1 to 10 carbon atoms, for instance a short-chain alkyl radical containing, for example, from 1 to 10 carbon atoms, in particular a methyl radical, or alternatively a phenyl group, preferably a methyl radical,
m is equal to 1 or 2, and
R′represents:
   an unsaturated aliphatic hydrocarbon-based group containing from 2 to 10 and preferably from 2 to 5 carbon atoms, for instance a vinyl group or a group —R″—CH═CHR′″ in which R″ is a divalent aliphatic hydrocarbon-based chain containing from 1 to 8 carbon atoms, bonded to the silicon atom and R′″ is a hydrogen atom or an alkyl radical containing from 1 to 4 carbon atoms, preferably a hydrogen atom; groups R′ that may be mentioned include vinyl and allylic groups and mixtures thereof; or
   an unsaturated cyclic hydrocarbon-based group containing from 5 to 8, carbon atoms, for instance a cyclohexenyl group.

Preferably, R′ is an unsaturated aliphatic hydrocarbon-based group, preferably a vinyl group.

According to one particular embodiment, the polyorgano-siloxane also comprises units of formula:

$${R}_{n} {SiO}_{\frac{( 4 - n )}{2}} \qquad ( II )$$

in which R is a group as defined above, and n is equal to 1, 2 or 3.

According to one variant, compound X may be a silicone resin comprising at least two ethylenic unsaturations, the said resin being capable of reacting with compound Y via hydrosilylation. Examples that may be mentioned include resins of MQ or MT type themselves bearing —CH═CH₂ unsaturated reactive ends.

These resins are crosslinked organosiloxane polymers.

The nomenclature of silicone resins is known under the name “MDTQ”, the resin being described as a function of the various siloxane monomer units it comprises, each of the letters M, D, T and Q characterizing a type of unit.

The letter M represents the monofunctional unit of formula (CH₃)₃SiO_{1/2}, the silicon atom being bonded to only one oxygen atom in the polymer comprising this unit.

The letter D means a difunctional unit (CH₃)₂SiO_{2/2} in which the silicon atom is bonded to two oxygen atoms.

The letter T represents a trifunctional unit of formula (CH₃)SiO_{3/2}.

In the units M, D and T defined above, at least one of the methyl groups may be substituted with a group R other than a methyl group, such as a hydrocarbon-based radical (especially alkyl) containing from 2 to 10 carbon atoms or a phenyl group, or alternatively a hydroxyl group.

Finally, the letter Q means a tetrafunctional unit SiO_{4/2} in which the silicon atom is bonded to four hydrogen atoms, which are themselves bonded to the rest of the polymer. Examples of such resins that may be mentioned include MT silicone resins such as poly(phenylvinylsilsesquioxane), for instance the product sold under the reference SST-3PV1 by the company Gelest.

Preferably, compounds X comprise from 0.01% to 1% by weight of unsaturated aliphatic groups.

Advantageously, compound X is chosen from polyorgano-polysiloxanes, especially those comprising the siloxane units (I) and optionally (II) described above.

Compound Y then comprises at least two free Si—H groups (hydrogenosilane groups).

Compound Y may be chosen advantageously from organo-siloxanes comprising at least one alkylhydrogenosiloxane unit having the following formula:

$${R}_{p} {SiO}_{\frac{( 3 - p )}{2}} \qquad ( III )$$

in which:

R represents a linear or cyclic monovalent hydrocarbon-based group containing from 1 to 30 carbon atoms, for instance an alkyl radical containing from 1 to 30 carbon atoms, preferably from 1 to 20 and better still from 1 to 10 carbon atoms, in particular a methyl radical, or alternatively a phenyl group, and p is equal to 1 or 2. Preferably, R is a hydrocarbon-based group, preferably methyl.

These organosiloxane compounds Y containing alkylhydrogenosiloxane units may also comprise units of formula:

$${R}_{n} {SiO}_{\frac{( 4 - n )}{2}} \qquad ( II )$$

as defined above.

Compound Y may be a silicone resin comprising at least one unit chosen from the units M, D, T and Q as defined above and comprising at least one Si—H group, such as the poly(methylhydridosilsesquioxanes) sold under the reference SST-3 MH1.1 by the company Gelest.

Preferably, these organosiloxane compounds Y comprise from 0.5% to 2.5% by weight of Si—H groups.

Advantageously, the radicals R represent a methyl group in formulae (I), (II) and (III) above.

Preferably, these organosiloxanes Y comprise end groups of formula (CH₃)₃SiO_{1/2}.

Advantageously, the organosiloxanes Y comprise at least two alkylhydrogenosiloxane units of formula (H₃C)(H)SiO and optionally comprise (H₃C)₂SiO units.

Such organosiloxane compounds Y containing hydrogenosilane groups are described, for example, in document EP 0 465 744.

According to one variant, compound X is chosen from organic oligomers or polymers (the term “organic” means compounds whose main chain is not silicone-based, preferably compounds comprising no silicon atoms) or from organic/silicone hybrid polymers or oligomers, the said oligomers or polymers bearing at least 2 reactive unsaturated aliphatic groups, compound Y being chosen from the hydrogenosiloxanes mentioned above.

Compound X, of organic nature, may then be chosen from vinyl or (meth)acrylic polymers or oligomers, polyesters, polyurethanes and/or polyureas, polyethers, perfluoropolyethers, polyolefins such as polybutene or polyisobutylene, dendrimers and organic hyperbranched polymers, or mixtures thereof.

In particular, the organic polymer or the organic part of the hybrid polymer may be chosen from the following polymers:
a) ethylenically unsaturated polyesters:

This is a group of polymers of polyester type containing at least two ethylenic double bonds, randomly distributed in the main polymer chain. These unsaturated polyesters are obtained by polycondensation of a mixture:
of linear or branched aliphatic or cycloaliphatic dicarboxylic acids especially containing from 3 to 50 carbon atoms, preferably from 3 to 20 and better from 3 to 10 carbon atoms, such as adipic acid or sebacic acid, of aromatic dicarboxylic acids especially containing from 8 to 50 carbon atoms, preferably from 8 to 20 and better from 8 to 14 carbon atoms, such as phthalic acids, especially terephthalic acid, and/or of dicarboxylic acids derived from ethylenically unsaturated fatty acid dimers such as the oleic or linoleic acid dimers described in patent application EP-A-959 066 (paragraph [0021]) sold under the names Pripol® by the company Uniqema or Empol® by the company Henkel, all these diacids needing to be free of polymerizable ethylenic double bonds,
of linear or branched aliphatic or cycloaliphatic diols especially containing from 2 to 50 carbon atoms, preferably from 2 to 20 and better from 2 to 10 carbon atoms, such as ethylene glycol, diethylene glycol, propylene glycol, 1,4-butanediol or cyclohexanedimethanol, of aromatic diols containing from 6 to 50 carbon atoms, preferably from 6 to 20 and better from 6 to 15 carbon atoms, such as bisphenol A and bisphenol B, and/or of diol dimers obtained from the reduction of fatty acid dimers as defined above, and
of one or more dicarboxylic acids or anhydrides thereof comprising at least one polymerizable ethylenic double bond and containing from 3 to 50 carbon atoms, preferably from 3 to 20 and better from 3 to 10 carbon atoms, such as maleic acid, fumaric acid or itaconic acid.
b) polyesters containing (meth)acrylate side groups and/or end groups:

This is a group of polymers of polyester type obtained by polycondensation of a mixture:
of linear or branched aliphatic or cycloaliphatic dicarboxylic acids especially containing from 3 to 50 carbon atoms, preferably from 3 to 20 and better from 3 to 10 carbon atoms, such as adipic acid or sebacic acid, of aromatic dicarboxylic acids especially containing from 8 to 50 carbon atoms, preferably from 8 to 20 and better from 8 to 14 carbon atoms, such as phthalic acids, especially terephthalic acid, and/or of dicarboxylic acids derived from ethylenically unsaturated fatty acid dimers such as the oleic acid or linoleic acid dimers described in patent application EP-A-959 066 (paragraph [0021]) sold under the names Pripol® by the company Uniqema or Empol® by the company Henkel, all these diacids needing to be free of polymerizable ethylenic double bonds,
of linear or branched aliphatic or cycloaliphatic diols especially containing from 2 to 50 carbon atoms, preferably from 2 to 20 and better from 2 to 10 carbon atoms, such as ethylene glycol, diethylene glycol, propylene glycol, 1,4-butanediol or cyclohexanedimethanol, of aromatic diols containing from 6 to 50 carbon atoms, preferably from 6 to 20 and better from 6 to 15 carbon atoms, such as bisphenol A and bisphenol B, and
of at least one ester of (meth)acrylic acid and of a diol or polyol containing from 2 to 20 carbon atoms and preferably from 2 to 6 carbon atoms, such as 2-hydroxyethyl (meth)acrylate, 2-hydroxypropyl (meth)acrylate or glycerol methacrylate.

These polyesters differ from those described above in point a) by the fact that the ethylenic double bonds are not located in the main chain but on side groups or at the end of the chains. These ethylenic double bonds are those of the (meth)acrylate groups present in the polymer.

Such polyesters are sold, for example, by the company UCB under the names Ebecryl® (Ebecryl® 450: molar mass 1600, on average 6 acrylate functions per molecule, Ebecryl® 652: molar mass 1500, on average 6 acrylate functions per molecule, Ebecryl® 800: molar mass 780, on average 4 acrylate functions per molecule, Ebecryl® 810: molar mass 1000, on average 4 acrylate functions per molecule, Ebecryl® 50 000: molar mass 1500, on average 6 acrylate functions per molecule)
c) polyurethanes and/or polyureas containing (meth)-acrylate groups, obtained by polycondensation
   of aliphatic, cycloaliphatic and/or aromatic diisocyanates, triisocyanates and/or polyisocyanates especially containing from 4 to 50 and preferably from 4 to 30 carbon atoms, such as hexamethylene diisocyanate, isophorone diisocyanate, toluene diisocyanate, diphenylmethane diisocyanate or isocyanurates of formula

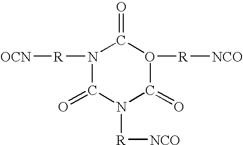

resulting from the trimerization of 3 molecules of diisocyanates OCN—R—CNO, in which R is a linear, branched or cyclic hydrocarbon-based radical comprising from 2 to 30 carbon atoms;
of polyols, especially of diols, free of polymerizable ethylenic unsaturations, such as 1,4-butanediol, ethylene glycol or trimethylolpropane, and/or of aliphatic, cycloaliphatic and/or aromatic polyamines, especially diamines, especially containing from 3 to 50 carbon atoms, such as ethylenediamine or hexamethylenediamine, and
of at least one ester of (meth)acrylic acid and of a diol or polyol containing from 2 to 20 carbon atoms and preferably from 2 to 6 carbon atoms, such as 2-hydroxyethyl (meth)acrylate, 2-hydroxypropyl (meth)acrylate or glycerol methacrylate.

Such polyurethanes/polyureas containing acrylate groups are sold, for example, under the name SR 368 (tris(2-hydroxyethyl) isocyanurate-triacrylate) or Craynor® 435 by the company Cray Valley, or under the name Ebecryl® by the company UCB (Ebecryl® 210: molecular mass 1500, 2 acrylate functions per molecule, Ebecryl® 230: molecular mass 5000, 2 acrylate functions per molecule, Ebecryl® 270: molecular mass 1500, 2 acrylate functions per molecule, Ebecryl® 8402: molecular mass 1000, 2 acrylate functions per molecule, Ebecryl® 8804: molecular mass 1300, 2 acrylate functions per molecule, Ebecryl® 220: molecular mass 1000, 6 acrylate functions per molecule, Ebecryl® 2220: molecular mass 1200, 6 acrylate functions per molecule, Ebecryl® 1290: molecular mass 1000, 6 acrylate functions per molecule, Ebecryl® 800: molecular mass 800, 6 acrylate functions per molecule).

Mention may also be made of the water-soluble aliphatic diacrylate polyurethanes sold under the names Ebecryl® 2000, Ebecryl® 2001 and Ebecryl® 2002, and the diacrylate polyurethanes in aqueous dispersion sold under the trade names IRR® 390, IRR® 400, IRR® 422 and IRR® 424 by the company UCB.
d) polyethers containing (meth)acrylate groups obtained by esterification, with (meth)acrylic acid, of the hydroxyl end groups of C₁₋₄ alkylene glycol homopolymers or copolymers, such as polyethylene glycol, polypropylene glycol, copolymers of ethylene oxide and of propylene oxide preferably having a weight-average molecular mass of less than 10,000, and polyethoxylated or polypropoxylated trimethylolpropane.

Polyoxyethylene di(meth)acrylates of suitable molar mass are sold, for example, under the names SR 259, SR 344, SR 610, SR 210, SR 603 and SR 252 by the company Cray Valley or under the name Ebecryl® 11 by UCB. Polyethoxylated trimethylolpropane triacrylates are sold, for example, under the names SR 454, SR 498, SR 502, SR 9035 and SR 415 by the company Cray Valley or under the name Ebecryl® 160 by the company UCB. Polypropoxylated trimethylolpropane triacrylates are sold, for example, under the names SR 492 and SR 501 by the company Cray Valley.
e) epoxyacrylates obtained by reaction between
   at least one diepoxide chosen, for example, from:
      (i) bisphenol A diglycidyl ether,
      (ii) a diepoxy resin resulting from the reaction between bisphenol A diglycidyl ether and epichlorohydrin,
      (iii) an epoxy ester resin containing α, ω-diepoxy end groups resulting from the condensation of a dicarboxylic acid containing from 3 to 50 carbon atoms with a stoichiometric excess of (i) and/or (ii), and
      (iv) an epoxy ether resin containing α, ω-diepoxy end groups resulting from the condensation of a diol containing from 3 to 50 carbon atoms with a stoichiometric excess of (i) and/or (ii),
      (v) natural or synthetic oils bearing at least 2 epoxide groups, such as epoxidized soybean oil, epoxidized linseed oil or epoxidized vernonia oil,
      (vi) a phenol-formaldehyde polycondensate (Novolac® resin), the end groups and/or side groups of which have been epoxidized,
         and
   one or more carboxylic acids or polycarboxylic acids comprising at least one ethylenic double bond in the α,β-position relative to the carboxylic group, for instance (meth)acrylic acid or crotonic acid or esters of (meth)acrylic acid and of a diol or polyol containing from 2 to 20 carbon atoms and preferably from 2 to 6 carbon atoms, such as 2-hydroxyethyl (meth)acrylate.

Such polymers are sold, for example, under the names SR 349, SR 601, CD 541, SR 602, SR 9036, SR 348, CD 540, SR 480 and CD 9038 by the company Cray Valley, under the names Ebecryl® 600, Ebecryl® 609, Ebecryl® 150, Ebecryl® 860 and Ebecryl® 3702 by the company UCB and under the names Photomer® 3005 and Photomer® 3082 by the company Henkel.
f) poly(C₁₋₅₀ alkyl (meth)acrylates), the said alkyl being linear, branched or cyclic, comprising at least two functions containing an ethylenic double bond borne by the hydrocarbon-based side chains and/or end chains.

Such copolymers are sold, for example, under the names IRR® 375, OTA® 480 and Ebecryl® 2047 by the company UCB.
g) polyolefins such as polybutene or polyisobutylene,
h) perfluoropolyethers containing acrylate groups obtained by esterification, for example with (meth)acrylic acid, of perfluoropolyethers bearing hydroxyl side groups and/or end groups.

Such α, ω-diol perfluoropolyethers are described especially in EP-A-1 057 849 and are sold by the company Ausimont under the name Fomblin® Z Diol.
i) hyperbranched dendrimers and polymers bearing (meth)acrylate or (meth)acrylamide end groups obtained, respectively, by esterification or amidation of hyperbranched dendrimers and polymers containing hydroxyl or amino end functions, with (meth)acrylic acid.

Dendrimers (from the Greek dendron=tree) are “arborescent”, i.e. highly branched, polymer molecules invented by D. A. Tomalia and his team at the start of the 1990s (Donald A. Tomalia et al., Angewandte Chemie, Int. Engl. Ed., Vol. 29, No. 2, pages 138-175). These are structures constructed about a central unit that is generally polyvalent. About this central unit are linked, in a fully determined structure, branched chain-extending units, thus giving rise to monodispersed symmetrical macromolecules having a well-defined chemical and stereochemical structure. Dendrimers of polyamidoamine type are sold, for example, under the name Starburst® by the company Dendritech.

Hyperbranched polymers are polycondensates, generally of polyester, polyamide or polyethyleneamine type, obtained from multifunctional monomers, which have an arborescent structure similar to that of dendrimers but are much less regular than dendrimers (see, for example, WO-A-93/17060 and WO 96/12754).

The company Perstorp sells hyperbranched polyesters under the name Boltorn®. Hyperbranched polyethylene-amines will be found under the name Comburst® from the company Dendritech. Hyperbranched poly(esteramides) containing hydroxyl end groups are sold by the company DSM under the name Hybrane®.

These hyperbranched dendrimers and polymers esterified or amidated with acrylic acid and/or methacrylic acid are distinguished from the polymers described in points a) to h) above by the very large number of ethylenic double bonds present. This high functionality, usually greater than 5, makes them particularly useful by allowing them to act as “crosslinking nodes”, i.e. sites of multiple crosslinking.

These dendritic and hyperbranched polymers may thus be used in combination with one or more of the polymers and/or oligomers a) to h) above.

1a—Additional Reactive Compounds

According to one embodiment, the compositions comprising compound X and/or Y may also comprise an additional reactive compound such as:
organic or mineral particles comprising at their surface at least 2 unsaturated aliphatic groups: mention may be made, for example, of silicas surface-treated, for example, with silicone compounds containing vinyl groups, for instance cyclotetramethyltetravinylsiloxane-treated silica,
silazane compounds such as hexamethyldisilazane.
   1b—Catalyst

The hydrosilylation reaction is advantageously performed in the presence of a catalyst that may be present in one or the other of the compositions comprising compound X and/or compound Y or in a separate composition, the catalyst preferably being platinum-based or tin-based.

Examples that may be mentioned include platinum-based catalysts deposited on a support of silica gel or charcoal powder (coal), platinum chloride, platinum salts and chloroplatinic acids.

Chloroplatinic acids in hexahydrate or anhydrous form, which are readily dispersible in organosilicone media, are preferably used.

Mention may also be made of platinum complexes such as those based on chloroplatinic acid hexahydrate and on divinyltetramethyldisiloxane.

The catalyst may be present in one or the other of the compositions useful in the present invention in a content ranging from 0.0001% to 20% by weight relative to the total weight of the composition comprising it. Polymerization inhibitors or retardants, and more particularly catalyst inhibitors, may also be introduced into the compositions of the invention, in order to increase the stability of the composition over time or to retard the polymerization. Non-limiting examples that may be mentioned include cyclic polymethylvinylsiloxanes, in particular tetravinyl-tetramethylcyclotetrasiloxane, and acetylenic alcohols, which are preferably volatile, such as methylisobutynol.

The presence of ionic salts, such as sodium acetate, in one and/or the other of the first and second compositions may have an influence on the rate of polymerization of the compounds.

As examples of combinations of compounds X and Y that react via hydrosilylation, mention may be made of the following references sold by the company Dow Corning: DC 7-9800 Soft Skin Adhesive Parts A & B, and also the following mixtures A and B prepared by Dow Corning:

### Mixture A:

| | | Content | |
|---|---|---|---|
| Ingredient (INCI name) | CAS No. | (weight %) | Function |
| Dimethylsiloxane, | 68083-19-2 | 55-95 | Polymer |
| Dimethylvinylsiloxy-terminated | | | |
| Silica Silylate | 68909-20-6 | 10-40 | Filler |
| 1,3-Diethenyl-1,1,3,3-Tetra- | 68478-92-2 | Trace | Catalyst |
| methyldisiloxane complexes | | | |
| Tetramethyldivinyldisiloxane | 2627-95-4 | 0.1-1 | Polymer |

### Mixture B:

| | | Content | |
|---|---|---|---|
| Ingredient (INCI name) | CAS No. | (weight %) | Function |
| Dimethylsiloxane, | 68083-19-2 | 55-95 | Polymer |
| Dimethylvinylsiloxy-terminated | | | |
| Silica Silylate | 68909-20-6 | 10-40 | Filler |
| Dimethyl, | 68037-59-2 | 1-10 | Polymer |
| Methylhydrogensiloxane, | | | |
| trimethylsiloxy-terminated | | | |

Advantageously, compounds X and Y are chosen from silicone compounds capable of reacting via hydrosilylation; in particular, compound X is chosen from polyorganosiloxanes comprising units of formula (I) described above and compound Y is chosen from organosiloxanes comprising alkylhydrogenosiloxane units of formula (III) described above. According to one particular embodiment, compound X is a polydimethylsiloxane containing vinyl end groups and compound Y is methylhydrogenosiloxane.

### 2—Compounds X and Y Capable of Reacting Via Condensation

According to this embodiment, compounds X and Y are capable of reacting via condensation, either in the presence of water (hydrolysis) by reaction of 2 compounds bearing alkoxysilane groups, or via “direct” condensation by reaction of a compound bearing alkoxysilane group(s) and a compound bearing silanol group(s) or by reaction of 2 compounds bearing silanol group(s).

When the condensation is performed in the presence of water, this water may in particular be ambient moisture, sweat or the water provided by an external source, for example premoistening of the keratin fibres (for example with a mister).

In this mode of reaction via condensation, compounds X and Y, which may be identical or different, may thus be chosen from silicone compounds whose main chain comprises at least two alkoxysilane groups and/or at least two silanol (Si—OH) groups, on the side and/or at the end of the chain.

In one advantageous embodiment, compounds X and/or Y are chosen from polyorganosiloxanes comprising at least two alkoxysilane groups. The term “alkoxysilane group” means a group comprising at least one —Si—OR portion, R being an alkyl group containing from 1 to 6 carbon atoms.

Compounds X and Y are especially chosen from poly-organosiloxanes comprising alkoxysilane end groups, more specifically those comprising at least 2 alkoxysilane end groups and preferably trialkoxysilane end groups.

These compounds X and/or Y preferably predominantly comprise units of formula:

R⁹sSiO(4-s)/2,

### (IV)

in which R⁹ independently represents a radical chosen from alkyl groups containing from 1 to 6 carbon atoms, phenyl and fluoroalkyl groups, and s is equal to 0, 1, 2 or 3. Preferably, R⁹ independently represents an alkyl group containing from 1 to 6 carbon atoms. Alkyl groups that may especially be mentioned include methyl, propyl, butyl, and hexyl, and mixtures thereof, preferably methyl or ethyl. A fluoroalkyl group that may be mentioned is 3,3,3-trifluoropropyl.

According to one particular embodiment, compounds X and Y, which may be identical or different, are polyorgano-siloxanes comprising units of formula: -(V) in which R⁹ is as described above, preferably R⁹ is a methyl radical, and f is such that the polymer advantageously has a viscosity at 250C ranging from 0.5 to 3000 Pa·s and preferably ranging from 5 to 150 Pa·s f is especially a number ranging from 2 to 5000, particularly from 3 to 3000 and more particularly from 5 to 1000.

These polyorganosiloxane compounds X and Y comprise at least 2 trialkoxysilane end groups per polymer molecule, the said groups having the following formula:

−ZSiR¹ₓ(OR)₃₋ₓ, (VI)

in which:

the radicals R independently represent a methyl, ethyl, n-propyl, isopropyl, n-butyl, sec-butyl or isobutyl group, preferably a methyl or ethyl group,

R¹ is a methyl or ethyl group,

x is equal to 0 or 1 and preferably x is equal to 0, and

Z is chosen from: divalent hydrocarbon-based groups not comprising any ethylenic unsaturation and containing from 2 to 18 carbon atoms (alkylene groups), combinations of divalent hydrocarbon-based radicals and of siloxane segments of formula (IX) below:

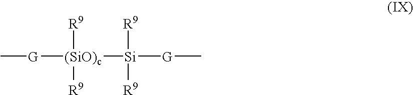

R⁹ being as described above, G is a divalent hydrocarbon-based radical not comprising any ethylenic unsaturation and containing from 2 to 18 carbon atoms and c is an integer ranging from 1 to 6.

Z and G may be chosen especially from alkylene groups such as ethylene, propylene, butylene, pentylene and hexylene, and arylene groups such as phenylene.

Preferably, Z is an alkylene group, and better still ethylene.

These polymers may contain on average at least 1.2 trialkoxysilane end groups or end chains per molecule, and preferably on average at least 1.5 trialkoxysilane end groups per molecule. Since these polymers may contain at least 1.2 trialkoxysilane end groups per molecule, some may comprise other types of end groups such as end groups of formula CH₂═CH═SiR⁹₂— or of formula R⁶₃—Si—, in which R⁹ is as defined above and each group R⁶ is independently chosen from groups R⁹ and vinyl. Examples of such end groups that may be mentioned include trimethoxysilane, triethoxysilane, vinyldimethoxysilane and vinylmethyloxyphenylsilane groups.

Such polymers are especially described in documents U.S. Pat. No. 3,175,993, U.S. Pat. No. 4,772,675, U.S. Pat. No. 4,871,827, U.S. Pat. No. 4,888,380, U.S. Pat. No. 4,898,910, U.S. Pat. No. 4,906,719 and U.S. Pat. No. 4,962,174, the content of which is incorporated into the present patent application by reference.

As compound X and/or Y, mention may be made in particular of the polymer of formula

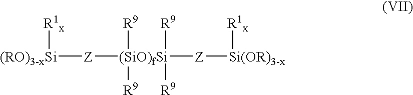

in which R, R¹, R⁹, Z, x and f are as described above.

Compounds X and/or Y may also comprise a mixture of polymer of formula (VII) above with polymers of formula (VIII) below:

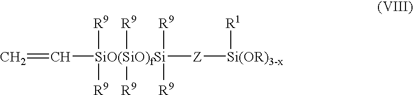

in which R, R¹, R⁹, Z, x and f are as described above.

When the polyorganosiloxane compound X and/or Y containing alkoxysilane group(s) comprises such a mixture, the various polyorganosiloxanes are present in contents such that the organosilyl end chains represent less than 40% and preferably less than 25% by number of the end chains.

The polyorganosiloxane compounds X and/or Y that are particularly preferred are those of formula (VII) described above. Such compounds X and/or Y are described, for example, in document WO 01/96450.

As indicated above, compounds X and Y may be identical or different.

According to one variant, one of the two reactive compounds X or Y is of silicone nature and the other is of organic nature. For example, compound X is chosen from organic oligomers or polymers or organic/silicone hybrid oligomers or polymers, the said polymers or oligomers comprising at least two alkoxysilane groups, and Y is chosen from silicone compounds such as the polyorganosiloxanes described above. In particular, the organic oligomers or polymers are chosen from vinyl, (meth)acrylic, polyester, polyamide, polyurethane and/or polyurea, polyether, polyolefin or perfluoro-polyether oligomers or polymers, and hyperbranched organic dendrimers and polymers, and mixtures thereof.

The organic polymers of vinyl or (meth)acrylic nature bearing alkoxysilane side groups may in particular be obtained via copolymerization of at least one organic vinyl or (meth)acrylic monomer with a (meth)acryloxy-propyltrimethoxysilane, a vinyltrimethoxysilane, a vinyltriethoxysilane, an allyltrimethoxysilane, etc.

Examples that may be mentioned include the (meth)acrylic polymers described in the document by Kusabe, M., Pitture e Verniei—European Coating; 12-B, pages 43-49, 2005, and especially the polyacrylates containing alkoxysilane groups referenced as MAX from Kaneka or those described in the publication by Probster, M., Adhesion-Kleben & Dichten, 2004, 481 (1-2), pages 12-14.

The organic polymers resulting from a polycondensation or a polyaddition, such as polyesters, polyamides, polyurethanes and/or polyureas, and polyethers, and bearing alkoxysilane side and/or end groups, may result, for example, from the reaction of an oligomeric prepolymer as described above with one of the following silane coreagents bearing at least one alkoxysilane group: aminopropyltrimethoxysilane, aminopropyltriethoxysilane, aminoethylaminopropyltrimethoxysilane, glycidoxypropyltrimethoxysilane, glycidoxypropyltriethoxysilane, epoxycyclohexylethyltrimethoxysilane, mercaptopropyltrimethoxysilane.

Examples of polyethers and polyisobutylenes containing alkoxysilane groups are described in the publication by Kusabe, M., Pitture e Verniei—European Coating; 12-B, pages 43-49, 2005. As examples of polyurethanes containing alkoxysilane end groups, mention may be made of those described in the document by Probster, M., Adhesion-Kleben & Dichten, 2004, 481 (1-2) pages 12-14 or those described in the document by Landon, S., Pitture e Verniei vol. 73, No. 11, pages 18-24, 1997 or in the document by Huang, Mowo, Pitture e Verniei vol. 5, 2000, pages 61-67; mention may be made especially of the polyurethanes containing alkoxysilane groups from OSI-WITCO-GE.

Polyorganosiloxane compounds X and/or Y that may be mentioned include resins of MQ or MT type themselves bearing alkoxysilane and/or silanol ends, for instance the poly(isobutylsilsesquioxane) resins functionalized with silanol groups sold under the reference SST-S7C41 (3 Si—OH groups) by the company Gelest.

2a—Additional Reactive Compound

One of the compositions that is useful in the present invention may also comprise an additional reactive compound comprising at least two alkoxysilane or silanol groups.

Mention may be made, for example, of one or more organic or mineral particles comprising at their surface alkoxysilane and/or silanol groups, for instance fillers surface-treated with such groups.

2b—Catalyst

The condensation reaction may be performed in the presence of a metal-based catalyst that may be present in one or the other of the compositions comprising X and/or Y or in a separate composition. The catalyst that is useful in this type of reaction is preferably a titanium-based catalyst.

Mention may be made especially of the tetraalkoxy-titanium-based catalysts of formula

Ti(OR²)_{y}(OR³)_{4-y},

in which R² is chosen from tertiary alkyl radicals such as tert-butyl, tert-amyl and 2,4-dimethyl-3-pentyl; R³ represents an alkyl radical containing from 1 to 6 carbon atoms, preferably a methyl, ethyl, n-propyl, isopropyl, n-butyl, sec-butyl or hexyl group and y is a number ranging from 3 to 4 and better still from 3.4 to 4.

The catalyst may be present in one or the other of the compositions that are useful in the present invention in a content ranging from 0.0001% to 20% by weight relative to the total weight of the composition(s) containing it.

2c—Diluent

The useful compositions comprising X and/or Y may also comprise a volatile silicone oil (or diluent) for reducing the viscosity of the composition.

This oil may be chosen from short-chain linear silicones such as hexamethyldisiloxane or octamethyltrisiloxane, and cyclic silicones such as octamethylcyclotetrasiloxane and decamethylcyclopentasiloxane, and mixtures thereof.

This silicone oil may represent from 5% to 95% and preferably from 10% to 80% by weight relative to the weight of each composition.

As examples of a combination of compounds X and Y bearing alkoxysilane groups and reacting via condensation, mention may be made of the combination of mixtures A′ and B′ below prepared by the company Dow Corning:

### Mixture A′:

| | | Content | |
|---|---|---|---|
| Ingredient (INCI name) | CAS No. | (weight %) | Function |
| Bis-trimethoxysiloxyethyl | PMN87176 | 25-45 | Polymer |
| tetramethyldisiloxyethyl | | | |
| dimethicone | | | |
| Silica silylate | 68909-20-6 | 5-20 | Filler |
| Disiloxane | 107-46-0 | 30-70 | Solvent |

### Mixture B′:

| | | Content | |
|---|---|---|---|
| Ingredient (INCI name) | CAS No. | (weight %) | Function |
| Disiloxane | 107-46-0 | 80-99 | Solvent |
| Tetra-t-butyl titanate | — | 1-20 | Catalyst |

It should moreover be noted that the identical compounds X and Y are combined in the mixture A′.

### 3—Crosslinking in the Presence of Peroxide:

This reaction is preferably performed by heating to a temperature of greater than or equal to 50° C., preferably greater than or equal to 80° C., which may be up to 120° C.

The identical or different compounds X and Y comprise in this case at least two —CH₃ side groups and/or at least two side chains bearing a —CH₃ group.

Compounds X and Y are preferably silicone compounds and may be chosen, for example, from high molecular weight non-volatile linear polydimethylsiloxanes, with a degree of polymerization of greater than 6, containing at least two —CH₃ side groups bonded to the silicon atom and/or at least two side chains bearing a —CH₃ group. Mention may be made, for example, of polymers described in the “Reactive Silicones” catalogue from the company Gelest Inc., Edition 2004, page 6, and especially vinylmethylsiloxane-dimethylsiloxane copolymers (also referred to as gums) with molecular weights ranging from 500 000 to 900 000 and a viscosity of greater than 2 000 000 cSt.

As peroxides that may be used in the context of the invention, mention may be made of benzoyl peroxide and 2,4-dichlorobenzoyl peroxide, and mixtures thereof.

According to one embodiment, the hydrosilylation reaction or the condensation reaction, or alternatively the crosslinking reaction in the presence of a peroxide, between compounds X and Y is accelerated by supplying heat, for example by raising the temperature of the system to between 25° C. and 180° C. The system will especially react on the skin.

In general, irrespective of the type of reaction via which compounds X and Y react together, the mole percentage of X relative to all of the compounds X and Y. i.e. the ratio X/(X+Y)×100, may range from 5% to 95%, preferably from 10% to 90% and better still from 20% to 80%.

Similarly, the mole percentage of Y relative to all of the compounds X and Y, i.e. the ratio Y/(X+Y)×100, may range from 5% to 95%, preferably from 10% to 90% and better still from 20% to 80%.

Compound X may have a weight-average molecular mass (Mw) ranging from 150 to 1 000 000, preferably from 200 to 800 000 and more preferably from 200 to 250 000.

Compound Y may have a weight-average molecular mass (Mw) ranging from 200 to 1 000 000, preferably from 300 to 800 000 and more preferably from 500 to 250 000.

Compound X may represent from 5% to 80% by weight and preferably from 10% to 50% by weight relative to the total weight of the composition.

Compound Y may represent from 0.5% to 80% by weight and preferably from 1% to 50% by weight relative to the total weight of the composition.

The ratio between the compounds X and Y may be varied so as to modify the rate of reaction and thus the rate of formation of the film, or alternatively so as to adapt the properties of the film formed (for example its adhesive properties) according to the desired application.

In particular, if X and Y are not the same, compounds X and Y may be present in an X/Y mole ratio ranging from 0.05 to 20 and better still from 0.1 to 10.

### Texturizing Agents

The composition applied to the hair fibres may also comprise one or more texturizing agents (fillers).

The term “texturizing agents” means mineral or synthetic, lamellar or non-lamellar, water-insoluble particles.

By way of example, these texturizing agents may be colloidal calcium carbonate, which may or may not be treated with stearic acid or stearate, silica such as fumed silicas, precipitated silicas and silicas treated to make them hydrophobic, ground quartz, alumina, aluminium hydroxide, titanium dioxide or diatomaceous earth.

Mention may also be made of talc, mica, kaolin, polyamide (Nylon®) powders (Orgasol from Atochem), polyethylene powders, tetrafluoroethylene polymer (Teflon®) powders, starch, boron nitride, polymer microspheres such as those of polyvinylidene chloride/acrylonitrile, for instance Expancel (Nobel Industrie) or of acrylic acid copolymers (Polytrap® from the company Dow Corning).

Synthetic silicas whose surface is modified with silicone compounds to make the surface hydrophobic are particularly preferred. These fillers are distinguished from each other by their surface properties, the silicone compounds used to treat the silica, and the way in which the surface treatment is performed.

Such agents make it possible to modify the viscosity of the formulation obtained with compounds X and/or Y.

Preferred fillers include silica, calcium carbonate and resin-based agents.

Examples that may be mentioned include the treated fillers Cab-O—Sil® TS-530, Aerosil® R8200 and Wacker HDX H2000.

These fillers may be present in a proportion of from 0 to 48% by weight, preferably 0.01% to 30% by weight and better still from 0.02% to 20% by weight relative to the total weight of the composition.

### Additives

The composition may also comprise at least one cosmetic additive.

Examples that may especially be mentioned include standard pigments, pigments with an effect (for example fluorescent, photochromic or thermochromic pigments, nacres or glitter flakes), antidandruff or anti-seborrhoeic agents, fragrances, hydroxy acids, electrolytes, preserving agents, silicone or non-silicone sunscreens, vitamins, provitamins such as panthenol, anionic or nonionic polymers, proteins, protein hydrolysates, 18-methyleicosanoic acid, synthetic oils such as polyolefins, mineral oils, plant oils, fluoro or perfluoro oils, natural or synthetic waxes, compounds of ceramide type, carboxylic acid esters, silicones, antioxidants, sequestrants, dispersants, conditioning agents, for instance cations, volatile or non-volatile, modified or unmodified silicones other than compounds X and Y, film-forming agents, ceramides, preserving agents, stabilizers and opacifiers, and also mixtures of these various products.

These additives are present in the composition according to the invention in proportions that may range from 0 to 20% by weight relative to the total weight of the composition. The precise amount of each additive is readily determined by a person skilled in the art depending on its nature and its function.

### Organic Solvents

The composition used in the context of the invention may comprise at least one organic solvent.

The term “organic solvent” means an organic substance that is liquid at a temperature of 25° C. and at atmospheric pressure (760 mmHg), which is capable of dissolving another substance without chemically modifying it.

It should be noted that the organic solvent(s) is (are) different from the compounds X and Y used in the context of the present invention.

The organic solvent(s) is (are) chosen, for example, from aromatic alcohols such as benzyl alcohol, phenoxy-ethanol and phenylethyl alcohol; liquid fatty alcohols, especially of C₁₀-C₃₀; C₁-C₆ alkanols comprising one or two free OH functions, such as ethanol, isopropanol, n-propanol, butanol, n-pentanol, 1,2-propanediol, 1,3-propanediol, 1-methoxy-2-propanol, 1-ethoxy-2-propanediol, 1,3- and 1,4-butanediol and 1,2-hexanediol; polyols and polyol ethers containing a free-OH function such as 2-butoxyethanol, propylene glycol, propylene glycol monomethyl ether, diethylene glycol monoethyl ether and monomethyl ether, ethylene glycol monoethyl ether, ethylene glycol monobutyl ether, neopentyl glycol, isoprene glycol, glycerol, glycol, dipropylene glycol, butylene glycol and butyl-diglycol; volatile silicones such as short-chain linear silicones such as hexamethyldisiloxane or octamethyltrisiloxane, cyclic silicones such as octamethylcyclotetrasiloxane, decamethylcyclopentasiloxane or dodecamethylcyclohexasiloxane, polydimethylsiloxanes optionally modified with alkyl and/or amine and/or imine and/or fluoroalkyl and/or carboxylic and/or betaine and/or quaternary ammonium functions; liquid modified polydimethylsiloxanes; mineral, organic or plant oils; alkanes and more particularly C₅-C₂₀ alkanes; liquid fatty acids; liquid fatty esters and more particularly liquid fatty alkyl benzoates or salicylates.

The organic solvent(s) is (are) preferably chosen from organic oils; silicones such as volatile silicones, amino or non-amino silicone gums or oils, and mixtures thereof; mineral oils; plant oils such as olive oil, castor oil, rapeseed oil, coconut oil, wheatgerm oil, apricot kernel oil, avocado oil, macadamia oil, apricot oil, safflower oil, candlenut oil, camellina oil, tamanu oil, lemon oil or organic compounds such as alkanes such as linear or branched C₅-C₂₀ alkanes, for instance isododecane, isohexadecane and isoparaffinic compounds of the type such as the products sold under the name Isopar E, acetone, methyl ethyl ketone, esters of liquid C₁-C₂₀ acids and of C₁-C₈ alcohols such as methyl acetate, butyl acetate, ethyl acetate and isopropyl myristate, dimethoxyethane, diethoxyethane, liquid C₁₀-C₃₀ fatty alcohols such as oleyl alcohol, liquid esters of fatty alcohols or of fatty acids such as C₁₀-C₃₀ fatty alkyl benzoates, and mixtures thereof; isononyl isononanoate, isostearyl malate, pentaerythrityl tetraisostearate, tridecyl trimellitate, polybutene oil, the mixture of cyclopentasiloxane (14.7% by weight)/polydimethylsiloxane dihydroxylated in the α and ω positions (85.3% by weight), or mixtures thereof.

According to one preferred embodiment, the organic solvent(s) is (are) chosen from silicones such as liquid polydimethylsiloxanes and modified liquid polydimethylsiloxanes, their viscosity at 25° C. being between 0.1 cSt and 1 000 000 cSt and more preferentially between 1 cSt and 30 000 cSt.

Mention will preferably be made of the following oils:
the mixture of α, ω-dihydroxylated polydimethylsiloxane/cyclopentadimethylsiloxane (14.7/85.3) sold by Dow Corning under the name DC 1501 Fluid;
the mixture of α, ω-dihydroxylated polydimethylsiloxane/polydimethylsiloxane sold by Dow Corning under the name DC 1503 Fluid;
the mixture of dimethicone/cyclopentadimethylsiloxane sold by Dow Corning under the name DC 1411 Fluid or that sold by Bayer under the name SF1214;
the cyclopentadimethylsiloxane sold by Dow Corning under the name DC245Fluid;
   and the respective mixtures of these oils.

The organic solvent(s) of the composition preferably represent(s) from 20% to 85% by weight relative to the total weight of the composition.

Preferably, the composition of the invention is anhydrous, i.e. it comprises less than 1% by weight of water relative to the total weight of the composition.

These compositions may be in various forms, such as lotions, aerosols, mousses or emulsions, and may be applied in the form of shampoos or hair conditioners.

In the case of aerosols, the composition of the invention may contain a propellant. The propellant is constituted of the compressed or liquefied gases usually used for the preparation of aerosol compositions. Air, carbon dioxide, compressed nitrogen or a soluble gas such as dimethyl ether, halogenated (in particular fluorinated) or non-halogenated hydrocarbons, and mixtures thereof, will preferentially be used.

Pocket aerosols containing one or more pockets may also be used.

Any device comprising multiple storage areas and a delivery system with one or more orifices enabling the products to be mixed at the outlet of this device may be used for the application of the products in general.

The composition according to the invention may be applied to dry or wet hair.

The composition comprising compound(s) X, Y may be obtained by extemporaneously mixing several compositions comprising one or more of the ingredients. Thus, it may be envisaged to mix a composition comprising at least compound X with a composition comprising at least compound Y. In the case where a catalyst or a peroxide is present, such an agent may be in one and/or the other of the abovementioned compositions or in a separate composition.

The invention is illustrated in greater detail by the examples described below.

Unless otherwise mentioned, the amounts indicated are mass percentages.

### EXAMPLE 1

In this example, the mixtures A′ and B′ below prepared by Dow Corning are used:

### Mixture A′:

| | | Contents | |
|---|---|---|---|
| Ingredient (INCI name) | CAS No. | (weight %) | Function |
| Bis-trimethylsiloxyethyl | PMN87176 | 25-45 | Polymer |
| tetramethyldisiloxyethyl | | | |
| dimethicone | | | |
| Silica silylate | 68909-20-6 | 5-20 | Filler |
| Disiloxane | 107-46-0 | 30-70 | Solvent |

### Mixture B′:

| | | Contents | |
|---|---|---|---|
| Ingredient (INCI name) | CAS No. | (weight %) | Function |
| Disiloxane | 107-46-0 | 80-99 | Solvent |
| Tetra-t-butyl titanate | — | 1-20 | Catalyst |

The following compositions are prepared:

### Composition 1

| | |
|---|---|
| Mixture A′ | 99.5% |
| Prestige Dazzling Red Gold (Eckart) | 0.5% |

### Composition 2

| | |
|---|---|
| Mixture B′ | 100% |

Compositions 1 and 2 are mixed together in a 10/1 weight ratio and then applied to wet hair.

The hair is dried in the open air for 30 minutes and then placed under a hood for 30 minutes.

Structured-lock hairstyles with glitter-flake effects, which are resistant to shampoo-washing several times, are obtained.

### EXAMPLE 2

In this example, the mixtures A and B below prepared by Dow Corning are used:

### Mixture A:

| | | Contents | |
|---|---|---|---|
| Ingredient (INCI name) | CAS No. | (weight %) | Function |
| Dimethylsiloxane, | 68083-19-2 | 55-95 | Polymer |
| dimethylvinylsiloxy- | | | |
| terminated | | | |
| Silica silylate | 68909-20-6 | 10-40 | Filler |
| 1,3-Diethenyl-1,1,3,3- | 68478-92-2 | Trace | Catalyst |
| tetramethyldisiloxane | | | |
| complexes | | | |
| Tetramethyldivinyldisiloxane | 2627-95-4 | 0.1-1 | Polymer |

### Mixture B:

| | | Contents | |
|---|---|---|---|
| Ingredient (INCI name) | CAS No. | (weight %) | Function |
| Dimethylsiloxane, | 68083-19-2 | 55-95 | Polymer |
| dimethylvinylsiloxy- | | | |
| terminated | | | |
| Silica silylate | 68909-20-6 | 10-40 | Filler |
| Dimethyl, methylhydrogen | 68037-59-2 | 1-10 | Polymer |
| siloxane, trimethylsiloxy- | | | |
| terminated | | | |

The following compositions are prepared:

### Composition 1

| | |
|---|---|
| Mixture A | 50% |
| Dow Corning 5225C Formulation Aid | 10% |
| Cyclopentadimethylsiloxane sold by Dow Corning | 39.8% |
| under the name DC245 Fluid | |
| Prestige Dazzling Red Gold (Eckart) | 0.2% |

### Composition 2

| | |
|---|---|
| Mixture B | 50% |
| Cyclopentadimethylsiloxane sold by Dow Corning | 50% |
| under the name DC245 Fluid | |

Compositions 1 and 2 are mixed together in a 1/1 weight ratio and then applied to wet or dry hair.

The hair is dried in the open air for 30 minutes and then placed under a hood for 30 minutes.

Structured-lock hairstyles with glitter-flake effects, which are resistant to shampoo-washing several times, are obtained.

### EXAMPLE 3

In this example, the mixtures A′ and B′ of Example 1 are used.

Two aerosol spray devices are prepared with the following compositions:

Aerosol 1: Composition 1

| | in g |
|---|---|
| Cyclopentadimethylsiloxane sold by Dow Corning | 45 |
| under the name DC245 Fluid | |
| Mixture A′ | 15 |
| Dimethyl ether | 40 |

Aerosol 2: Composition 2

| | in g |
|---|---|
| Cyclopentadimethylsiloxane sold by Dow Corning | 58.5 |
| under the name DC245 Fluid | |
| Mixture B′ | 1.5 |
| Dimethyl ether | 40 |

A structured-lock hairstyle is constructed with aerosol 1, and aerosol 2 is then applied to finish.

The hairstyle has very good hold.

The structured locks are resistant to shampoo-washing several times.

1. Process for treating human keratin fibres, such as the hair, to obtain structured hairstyles, in which a composition comprising at least 5% by weight of a compound X, at least 0.5% by weight of a compound Y, is applied, at least one of the compounds X or Y being a silicone compound, the said compounds X and Y being capable of reacting together via a hydrosilylation reaction, or via a condensation reaction, or via a crosslinking reaction in the presence of a peroxide.

2-41. (canceled)
